(19) United States
(12) Patent Application Publication
(10) Pub. No.: US 2025/0057514 A1
(43) Pub. Date: Feb. 20, 2025

(54) MEDICAL CUTTING DEVICES AND METHODS OF USE THEREOF

(71) Applicant: George G. KIHICZAK, New York, NY (US)

(72) Inventor: George G. KIHICZAK, New York, NY (US)

(21) Appl. No.: 18/712,578

(22) PCT Filed: Dec. 6, 2022
(86) PCT No.: PCT/US2022/081035

§ 371 Date: May 22, 2024

Related U.S. Application Data
(60) Provisional application No. 63/286,174, filed on Dec. 6, 2021.

(51) Int. Cl. A61B 10/02; A61B 17/3211

(52) U.S. Cl. CPC A61B 10/0266; A61B 17/3211

(57) ABSTRACT

Disclosed in embodiments described here are cutting devices, such as but not limited to, medical or surgical blades and scalpels, and methods of using the cutting devices of the description that can be used to obtain a skin sample from a subject or to remove a skin sample from a subject.

### RELATED APPLICATIONS

This International application, filed herewith, claims priority to U.S. Provisional Application No. 63/286,174, filed Dec. 6, 2021, the entirety of which is incorporated herein by reference for all purposes.

### FIELD OF THE DISCLOSURE

The present disclosure relates to surgical tools. More specifically, the present disclosure relates to a novel medical blade and scalpel comprising the medical blade as described here used in a surgical procedure for experimental, diagnostic, prophylactic, therapeutic purposes, or combinations thereof.

### SUMMARY

In accordance with the foregoing objectives and others, the present disclosure provides in some embodiments, medical cutting devices and methods of use thereof, where medical cutting devices can comprise of, consist essentially of, or consist of medical blades and scalpels comprising, consisting essentially of, or consisting of medical cutting devices and a handle. In some aspects, the disclosure provides a medical blade, comprising, consisting essentially of, or consists of: a cutting portion, where the cutting portion comprises, consists essentially of, or consists of a symmetrical arcuate portion, where the symmetrical arcuate portion comprises, consists essentially of, consists of a first end of the symmetrical arcuate portion, a second end of the symmetrical arcuate portion, and an arc therebetween, where the first end of the symmetrical arcuate portion and the second end of the symmetrical arcuate portion are on an axis, where the axis is the same axis, where the second end of the symmetrical arcuate portion is distal to the first end of the symmetrical arcuate portion, where the symmetrical arcuate portion comprises, consists essentially of, or consists of a width extending on the axis between the first end of the symmetrical arcuate portion and the second end of the symmetrical arcuate portion, where the arc comprises, consists essentially of, or consists of a rise extending from the center of the width to the center of the arc, where the symmetrical arcuate portion comprises, consists essentially of, or consists of: (i) a convex side; (ii) a concave side; (iii) a top edge; and (iv) a bottom edge, where the bottom edge comprises, consists essentially of, or consists of a cutting edge, where the symmetrical arcuate portion comprises, consists essentially of, or consists of a height extending between the top edge and the bottom edge, where the cutting portion is configured to be fixed (or fixedly configured, operatively connectable) at the first end of the symmetrical arcuate portion. In some aspects, the medical blade described here can further comprise, consist essentially of, or consist of: a body portion, where the body portion comprises, consists essentially of, or consists of a first end (proximal) and a second end (distal), where the body portion is linear (e.g., essentially linear), extending along a longitudinal axis between the first end of the body portion and the second end of the body portion, where the second end of the body portion is distal to the first end of the body portion, where the cutting portion is configured to be fixed at the second end of the body portion, where the body portion is configured to be fixed at the first end of the body portion. In other aspects, the medical blades described here, where the medical blade comprises, consists essentially of, or consists of a cutting portion or a cutting portion and a body portion, and the medical blade can be a surgical blade.

In other aspects of the disclosure, the medical blade of the disclosure can comprise, consist essentially of, or consist of a shape selected from the group consisting of: a parabolic shape, a semi-elliptical shape, a semi-circular shape, a semi-ovoid shape, a C-shape, a J-shape, and a question mark shape.

In some aspects, any one of the medical blades of the disclosure can comprise, consist essentially of, or consist of a first end of the symmetrical arcuate portion and a second end of the symmetrical arcuate portion which lie on an axis of a width extending between the first end of the symmetrical arcuate portion and the second end of the symmetrical arcuate portion. In various aspects, the axis on which the first and the second ends of the symmetrical arcuate portion is a longitudinal axis. In other aspects, the axis of the width extending between the first end of the symmetrical arcuate portion and the second end of the symmetrical arcuate portion is perpendicular to the longitudinal axis. Some aspects provide the medical blade of the disclosure comprising, consisting essentially of, or consisting of at the first end of the symmetrical arcuate portion an attachment region. In other aspects, the first end of the body portion can comprise, consist essentially of, or consist of an attachment region. Various aspects provide for an attachment region of the symmetrical arcuate portion or the body portion, which comprises, consists essentially of, or consists of at least one aperture. In some aspects, the attachment region of the symmetrical arcuate portion or the body portion is configured for attachment to a handle, for example a handle of a surgical device or scalpel.

Some aspects of the disclosure provide for medical blades comprising, consisting essentially of, or consists of a width along the axis positioned between a first end of a symmetrical arcuate portion and a second end of a symmetrical arcuate portion, where the width comprises, consists essentially of, or consists of 0.5 mm or greater; the rise having a length extending from the center of the width to the center of the arc of the symmetrical arcuate portion of the medical blade, where the rise comprises, consists essentially of, or consists of a length of 0.5 mm or greater; a height extending between the top edge and the bottom edge of the symmetrical arcuate portion of the medical blade described here, where the height comprises, consists essentially of, or consists of 0.1 mm or greater. In other aspects of the disclosure, the symmetrical arcuate portion of the cutting portion of the medical blade of the disclosure comprises, consists essentially of, or consists of a thickness between the concave side and the convex side, where the thickness is 0.01 mm or greater. Some aspects of the disclosure provide for a medical blade described here that is composed of a material selected from the group consisting of: metal (e.g., stainless steel, high carbon steel, tempered steel), ceramic (e.g., zirconium, zirconium dioxide), titanium, diamond, sapphire, obsidian, combinations of metals, and metal ceramic alloys (e.g., cermet).

In other aspects, the cutting device comprises, consists essentially of, or consists of a scalpel, comprising, consists essentially of, or consists of: the medical blade of the disclosure; and a handle, where the handle is linear along a longitudinal axis, where the handle comprises, consists essentially of, or consists of a first end (proximal) and a second end (distal), where the second end of the handle is fixedly attached (e.g., temporarily, permanently; non-folding, non-sliding) to the medical blade at the attachment region (wherein the second end of the handle is fixedly attached at the attachment region of the medical blade). Some aspects of the disclosure provide a scalpel where the second end of the handle comprises, consists essentially of, or consists of at least one projection (e.g., stud, screw, pin, elongated projection) that is configured to fasten in the at least one aperture (e.g., keyhole, slotted hole, opening) at the first end of the medical blade. Other aspects provide a scalpel where the second end of the handle comprises, consists essentially of, or consists of at least aperture (e.g., keyhole, slotted hole, opening) that is configured to fasten in the at least one projection (e.g., stud, screw, pin, elongated projection).

In some aspects, the disclosure can be directed to a method of obtaining a skin sample from a subject, comprising, consisting essentially of, or consisting of: moving the cutting portion of any medical blade or of any scalpel described here through the surface of skin of a subject to obtain a skin sample. The moving action comprises, consists essentially of, or consists of moving or rotating the scalpel around the longitudinal axis or around an axis perpendicular to the longitudinal axis. In other aspects, the method of the disclosure comprises, consists essentially of, or consists of moving the cutting portion of the scalpel described here in a downward and an upward motion to remove a skin sample from the subject.

### BRIEF DESCRIPTION OF FIGURES

FIG. 1A shows an exemplary medical blade (100) with a cutting portion comprising a top edge (110) and a bottom edge (120) of a symmetrical arcuate portion (130), a first end of the symmetrical arcuate portion (Fa) and a second end of the symmetrical arcuate portion (Sa) positioned on a w-w axis, where the Sa is distal to the Fa, where a width (w) on the w-w axis between the Fa and the Sa has a center point, where a rise (r) is the distance from the center point of the width on the w-w axis to the bottom edge of the symmetrical arcuate portion, and a height (h) is the distance between the top edge (110) and the bottom edge (120) of the symmetrical arcuate portion (130).

FIG. 1B shows an exemplary medical blade (100) with a top edge (110) that tapers down to the bottom edge (120) from a center point of the symmetrical arcuate portion (130) to each of a first end of the symmetrical arcuate portion (Fa) and a second end of the symmetrical arcuate portion (Sa) positioned on a w-w axis, where a rise (r) is the distance from the center point of the width on the w-w axis to the bottom edge of the symmetrical arcuate portion, and a height (h) is the distance between the top edge (110) and the bottom edge (120) of the symmetrical arcuate portion (130).

FIG. 1C shows an exemplary medical blade (100) having a rise of an arc of a symmetrical arcuate portion (130) of a medical blade comprising a length from a point on the w-w axis (r_{w}) to a point at the bottom edge (120) of the arc furthest from the w-w axis, and the top edge (110) of the medical blade (100). An arc portion comprising a width from Fa to r_{w} forming a first width (w₁) and a width from a second end of the symmetrical arcuate portion (Sa) to r_{w} forming a second width (w₂). In this exemplary medical blade, w₁ is greater than w₂. The arc portion from the point at the bottom edge (120) of the exemplary medical blade extending from r_{w} to Fa comprises an angle of X₁° and another arc portion from the point at the bottom edge (120) of the exemplary medical blade extending from r_{w} to Sa comprises an angle of X₂.

FIG. 2 shows atop view looking down on the top edge (210) of a symmetrical arcuate portion (230) of a cutting portion of an exemplary medical blade (200). The dashed line represents the bottom edge (220), where the thickness (t) of the top edge (210) of the symmetrical arcuate portion (230) is shown.

FIGS. 3A-3B show illustrations of exemplary medical blades. FIG. 3A presents a medical blade (300) with a cutting portion comprising atop edge (310) and a bottom edge (320) of a symmetrical arcuate portion (330) and a body portion (340), where the first end of the symmetrical arcuate portion (Fa) and second end of the symmetrical arcuate portion (Sa) are positioned on a w-w axis, where the body portion is positioned along an axis perpendicular to the w-w axis or positioned along a B-B axis, and the body portion comprises a first end of the body portion (Fb) and the second end of the body portion (Sb) distal to the Fb. FIG. 3B shows an angled view of an exemplary medical blade (300) with a cutting portion comprising a top edge (310) and a bottom edge (320) of a symmetrical arcuate portion (330), and a body portion (340) containing an attachment region (A) (e.g., female receiving aperture: slotted hole).

FIGS. 4A-4B show exemplary scalpels (450) comprising a medical blade (400) comprising a cutting portion. FIG. 4A shows a cutting portion comprising a symmetrical arcuate portion (430) and body portion, where the first end of the symmetrical arcuate portion (Fa) and the second end of the symmetrical arcuate portion (Sa) are positioned along the w-w axis, and the Fa is fixedly attached to a handle (460) comprising a first end of the handle (Fh) and a second end of the handle (Sh) distal to the Fh along an H-H axis, where the Fa is fixedly attached to the second end of the handle (Sh) at an attachment region (A). FIG. 4B shows a cutting portion comprising a symmetrical arcuate portion, where the first end of the symmetrical arcuate portion (Fa) and the second end of the symmetrical arcuate portion (Sa) positioned along the H-H axis, and the Fa is fixedly attached to a handle comprising a first end of the handle (Fh) and a second end of the handle (Sh) distal to the Fh along the H-H axis, where the Fa is fixedly attached to the second end of the handle (Sh) at an attachment region (A).

FIGS. 5A-5B show exemplary scalpels (550) comprising a medical blade comprising a cutting portion. FIG. 5A shows a medical blade comprising a cutting portion with a symmetrical arcuate portion (530) and a body portion (540), where the first end of the symmetrical arcuate portion (Fa) and the second end of the symmetrical arcuate portion (Sa) are positioned along the w-w axis, where the body portion (540) is positioned along an axis perpendicular to the w-w axis or positioned along an H-H axis, where the body portion comprises a first end of the body portion (Fb) and the second end of the body portion (Sb) distal to the Fb, and the Fa is fixedly attached to the second end of the body portion (Sb), where the first end of the body portion (Fb) is fixedly attached to a handle (560) comprising a first end of the handle (Fh) and a second end of the handle (Sh) distal to the Fh along an H-H axis, where the Fb is fixedly attached to the second end of the handle (Sh) at an attachment region (A). FIG. 5B shows a medical blade with a cutting portion comprising a symmetrical arcuate portion (530) and a body portion (540), where the first end of the symmetrical arcuate portion (Fa) and the second end of the symmetrical arcuate portion (Sa) are positioned along the H-H axis, where the body portion (540) is positioned along the H-H axis, and the body portion (540) comprises a first end of the body portion (Fb) and a second end of the body portion (Sb) distal to the Fb, and the Fb is fixedly attached to a handle (560) comprising a first end of the handle (Fh) and a second end of the handle (Sh) distal to the Fh along the H-H axis, where the Fb is fixedly attached to the second end of the handle (Sh) at an attachment region (A).

### DETAILED DESCRIPTION

Detailed embodiments of the present disclosure are disclosed herein; however, it is to be understood that the disclosed embodiments are merely illustrative of the disclosure that can be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, the term “about” a number can refer to that number plus or minus 10% of that number.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1 mm to 3 mm specifically discloses a length of 0.1 mm, 1 mm, 1.5 mm, 2.0 mm, 2.5 mm, and 3 mm. Additionally, a range of 0.1 mm to 3 mm includes subsets of the original range including from 0.5 mm to 2.5 mm, from 1 mm to 3 mm, from 0.1 mm to 2.5 mm, etc.

By “consist” it is meant that the devices and methods of the disclosure include only the listed components and steps. By “consisting essentially” is meant with any other components present which do not affect the operation of the embodiments disclosed herein.

It is to be understood that terms such as “left”, “right” “top”, “bottom”, “front”, “rear”, “side”, “height”, “length”, “width”, “upper”, “lower”, “interior”, “exterior”, “inner, “outer” and the like as can be used herein to describe points of reference and do not limit the present invention to any particular orientation or configuration.

By “line of symmetry” as used herein is meant an axis that divides an object or, for example, an arcuate portion of a medical blade described here, into mirror images. In some embodiments, the line of symmetry is the y-axis or height (h) of the medical blade of the disclosure.

### Medical Blade

### Cutting Portion

The disclosure as described here provides a cutting device, such as a medical blade, where the medical blade can be a surgical blade used for surgical purposes. Also described is a scalpel comprising, consisting essentially of, or consisting of the medical blade and a handle fixedly attached thereto, methods of using the medical blade or the scalpel of the disclosure, methods of obtaining a skin sample using the medical blade or the scalpel of the disclosure, and use of the medical blade or the scalpel of the disclosure for obtaining a skin sample.

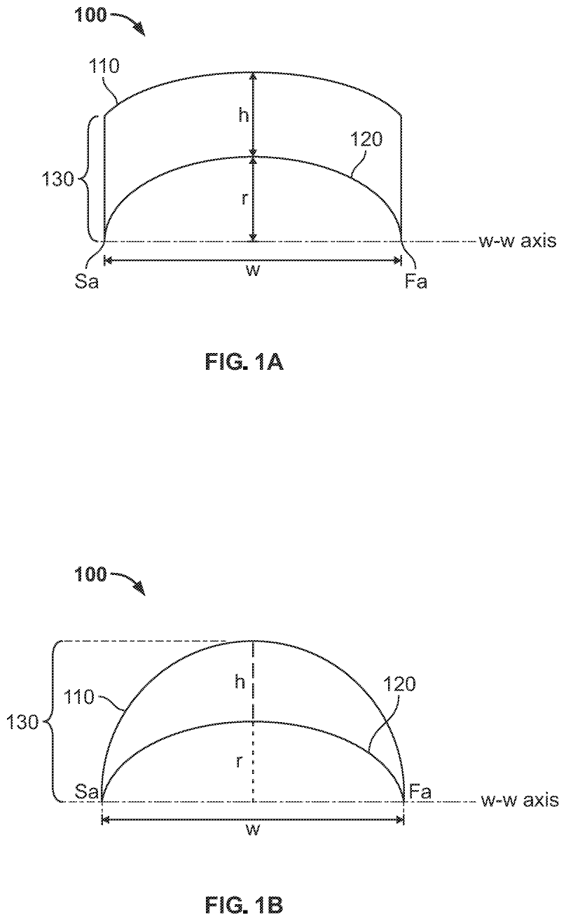

FIG. 1A shows an exemplary medical blade (100) of the disclosure, where the medical blade described here can comprise a cutting portion that can be a symmetrical arcuate or have a symmetrical arcuate portion, where the height is uniform from a first end to a second end of the symmetrical arcuate portion. FIG. 1B shows an exemplary medical blade of the disclosure, where the medical blade described here can comprise a cutting portion that can be a symmetrical arcuate or have a symmetrical arcuate portion, where the height is tapered from a center point of the symmetrical arcuate portion to each of a first end and a second end of the symmetrical arcuate portion. As used here, the “symmetrical arcuate portion” means a curvature or arc where both sides around a central point or axis (e.g., line of symmetry) are identical, essentially identical, or substantially identical. Some embodiments of the disclosure are directed to a cutting portion comprising a symmetrical arcuate portion configured around a line of symmetry forming two arc portions, where each arc portion is located on one side or an opposite side of the line of symmetry. An arc portion on one side of the line of symmetry of the symmetrical arcuate portion comprises a number of degrees (X₁) from the line of symmetry and an arc portion on an opposite side of the line of symmetry of the symmetrical arcuate portion comprises a number of degrees (X₂), where in some examples, X₁=X₂. For example, an arc portion on one side of the line of symmetry of a symmetrical arcuate portion comprises X₁ degrees from the line of symmetry and an arc portion on the opposite side of the line of symmetry of the symmetrical arcuate portion comprises X₂ degrees from the line of symmetry, where X₁ and X₂ are equal or have the same number of degrees. In further examples, an arc portion comprises X₁ degrees from the line of symmetry that can be 0.1% or greater than X₂ degrees (e.g., 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 30%, 40%, 50%, 60%, 70%, 80%, 90%, 100%); X₁ degrees can be 100% or less than X₂ degrees (e.g., 95%, 85%, 75%, 65%, 55%, 45%, 35%, 25%, 19%, 17%, 15%, 13%, 11%); or X₁ degrees can be 0.1%-100% of X₂ degrees (e.g., 0.2%-99%; 0.3%-98%; 0.4%-97%; 0.5%-96%, 0.6%-95%; 0.7%-94%; 0.8%-93%; 0.9%-92%; 1%-91%; 2%-90%; 3%-89%; 4%-88%; 5%-87%; 6%-86%; 7%-85%; 8%-84%; 9%-83%; 10%-82%; 15%-81%; 20%-80%; 22%-79%; 23%-78%; 24%-77%; 25%-76%; 26%-75%; 27%-74%; 28%-73%; 29%-72%; 30%-71%; 31-%-70%; 32%-69%; 33%-68%; 34%-67%; 35%-66%; 36%-65%; 37%-64%; 38%-63%; 39%-62%; 40%-61%; 41%-60%; 42%-59%; 43%-58%; 44%-57%; 45%-56%; 46%-55%; 47%-54%; 48%-53%; 49%-52%; 50%-51%). In some examples, an arc portion comprises X₂ degrees from the line of symmetry that can be 0.1% or greater than X₁ degrees (e.g., 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12, 14%, 16, 18%, 20%, 30%, 40%, 50%, 60%, 70%, 80%, 90%, 100%); X₂ degrees can be 100% or less than X₁ degrees (e.g., 95%, 85%, 75%, 65%, 55%, 45%, 35%, 25%, 19%, 17%, 15%, 13%, 11%); or X₂ degrees can be 0.1%-100% of X₁ degrees (e.g., 0.2%-99%; 0.3%-98%; 0.4%-97%; 0.5%-96%, 0.6%-95%; 0.7%-94%; 0.8%-93%; 0.9%-92%; 1%-91%; 2%-90%; 3%-89%; 4%-88%; 5%-87%; 6%-86%; 7%-85%; 8%-84%; 9%-83%; 10%-82%; 15%-81%; 20%-80%; 22%-79%; 23%-78%; 24%-77%; 25%-76%; 26%-75%; 27%-74%; 28%-73%; 29%-72%; 30%-71%; 31-%-70%; 32%-69%; 33%-68%; 34%-67%; 35%-66%; 36%-65%; 37%-64%; 38%-63%; 39%-62%; 40%-61%; 41%-60%; 42%-59%; 43%-58%; 44%-57%; 45%-56%; 46%-55%; 47%-54%; 48%-53%; 49%-52%; 50%-51%). See, e.g., FIG. 1C.

The symmetrical arcuate portion can comprise a first end of the symmetrical arcuate portion (Fa), a second end of the symmetrical arcuate portion (Sa), and an arc therebetween, where the first end of the symmetrical arcuate portion and the second end of the symmetrical arcuate portion are on the same axis (w-w axis), and the second end of the symmetrical arcuate portion is distal to the first end of the symmetrical arcuate portion. Some embodiments provide for the described symmetrical arcuate portion comprising (i) a convex side, (ii) a concave side, (iii) a top edge, and (iv) a bottom edge, where the symmetrical arcuate portion comprises a height (h) (e.g., z-axis) extending between the top edge and the bottom edge. In some aspects, the bottom edge comprises a cutting edge or the edge of the medical blade, where the edge is sharp or sharp enough to cut through at least one of the various layers of skin.

The symmetrical arcuate portion comprises a width (w) having a length extending on the axis (w-w axis) between the first end of the symmetrical arcuate portion (Fa) and the second end of the symmetrical arcuate portion (Sa). The arc of the symmetrical arcuate portion comprises a rise (r) having a length extending from the center of the width (w) to the center of the arc, or the point on the arc furthest away from the w-w axis between the first (Fa) and the second ends (Sa) of the symmetrical arcuate portion or the width. For example, if the symmetrical arcuate portion is a semi-circle, then the width would be the diameter (e.g., x-axis) and the rise (r) would be the radius (e.g., y-axis). In some examples, the medical blade of the disclosure comprises a cutting portion of the symmetrical arcuate portion described here having a width (w) (e.g., x-axis), a rise (r) (e.g., y-axis), and a height (h) (e.g., z-axis). Some examples provide for the described cutting portion, where the cutting portion can be: fixed, configured to be fixed, fixedly configured, or operatively connectable at the first end of the symmetrical arcuate portion. In some aspects, the width has a length of about the rise or greater than about the rise; the width has a length of about the height or greater than about the height; the rise has a length of about the width or greater than about the width; the rise has a length of about the height or greater than about the height; the height has a length of about the width or less than about the width; or the height has a length of about the rise or greater than about the rise.

In additional examples of the disclosure, a rise of an arc of a symmetrical arcuate portion of a medical blade can comprise a length from a point on the w-w axis (r_{w}) to a point at the bottom edge of the arc furthest from the w-w axis. Some aspects of this embodiment provide a width from a first end of the symmetrical arcuate portion (Fa) to r_{w} forming a first width (w₁) and a width from a second end of the symmetrical arcuate portion (Sa) to r_{w} forming a second width (w₂) (see, e.g., FIG. 1C). In some embodiments of the symmetrical arcuate portion of the disclosure, w₁ can be the same as w₂. Other embodiments of the symmetrical arcuate portion of the disclosure, w₁ can be greater than w₂. Further embodiments of the symmetrical arcuate portion of the disclosure, w₁ can be less than w₂. In some aspects, w₁ can be 0.1% or greater than w₂ (e.g., 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 30%, 40%, 50%, 60%, 70%, 80%, 90%, 100%); 100% or less than w₂ (e.g., 95%, 85%, 75%, 65%, 55%, 45%, 35%, 25%, 19%, 17%, 15%, 13%, 11%); 1%-100% of w₂ (e.g., 2%-99%; 3%-98%; 4%-97%; 5%-96%, 6%-95%; 7%-94%; 8%-93%; 9%-92%; 10%-91%; 11%-90%; 12%-89%; 13%-88%; 14%-87%; 15%-86%; 16%-85%; 17%-84%; 18%-83%; 19%-82%; 20%-81%; 21%-80%; 22%-79%; 23%-78%; 24%-77%; 25%-76%; 26%-75%; 27%-74%; 28%-73%; 29%-72%; 30%-71%; 31-%-70%; 32%-69%; 33%-68%; 34%-67%; 35%-66%; 36%-65%; 37%-64%; 38%-63%; 39%-62%; 40%-61%; 41%-60%; 42%-59%; 43%-58%; 44%-57%; 45%-56%; 46%-55%; 47%-54%; 48%-53%; 49%-52%; 50%-51%). Other embodiments of the symmetrical arcuate portion of the disclosure, w₂ can be greater than w₁. Further embodiments of the symmetrical arcuate portion of the disclosure, w₂ can be less than w₁. In other examples of the symmetrical arcuate portion of the disclosure, w₂ can be 0.10% or greater than w₁ (e.g., 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%, 30%, 40%, 50%, 60%, 70%, 80%, 90%, 100%); 100% or less than w₁ (e.g., 95%, 85%, 75%, 65%, 55%, 45%, 35%, 25%, 19%, 17%, 15%, 13%, 11%); 1%-100% of w₁ (e.g., 2%-99%; 3%-98%; 4%-97%; 5%-96%, 6%-95%; 7%-94%; 8%-93%; 9%-92%; 10%-91%; 11%-90%; 12%-89%; 13%-88%; 14%-87%; 15%-86%; 16%-85%; 17%-84%; 18%-83%; 19%-82%; 20%-81%; 21%-80%; 22%-79%; 23%-78%; 24%-77%; 25%-76%; 26%-75%; 27%-74%; 28%-73%; 29%-72%; 30%-71%; 31-%-70%; 32%-69%; 33%-68%; 34%-67%; 35%-66%; 36%-65%; 37%-64%; 38%-63%; 39%-62%; 40%-61%; 41%-60%; 42%-59%; 43%-58%; 44%-57%; 45%-56%; 46%-55%; 47%-54%; 48%-53%; 49%-52%; 50%-51%).

In some embodiments, the medical blade of the description comprises a symmetrical arcuate portion that can include, but not limited to, half of a circle, half of an ellipse, half of an oval, half of a hyperbola, a parabola, or any other shape that can provide a symmetrical arc. Non-limiting examples of the medical blades of the disclosure include those having shapes selected from the group consisting of: a parabolic shape, a semi-elliptical or a semi-ovoid shape, a semi-circular shape, a semi-ovoid shape, a C-shape, a J-shape, and a question mark shape.

Some aspects provide medical blades of the disclosure, where the axis of the width (e.g., w-w axis) extending between the first end of the symmetrical arcuate portion (Fa) and the second end of the symmetrical arcuate portion (Sa) can be a longitudinal axis (e.g., y-axis, B-B axis, H-H axis) or an axis perpendicular, or essentially perpendicular “w-w axis” (e.g., ˜80°-˜100°, ˜85°, ˜90°, ˜95° from the longitudinal axis) to the longitudinal axis (e.g., y-axis, B-B axis, H-H axis). In some instances, the width (w) may be along the longitudinal axis (e.g., y-axis, B-B axis, H-H axis) or the width (w) may be along an axis (e.g., x-axis) that is perpendicular to or 90° from the longitudinal axis (e.g., B-B axis, H-H axis).

In some embodiments of the description, the first end of the symmetrical arcuate portion (Fa) can comprise an attachment region. Various examples provide an attachment region comprising, consisting essentially of, or consisting of a length of 1 cm or greater (e.g., 1.1; 1.2; 1.3; 1.4; 1.5; 1.6; 1.7; 1.8; 1.9; 2; 2.1; 2.2; 2.3; 2.4; 2.5; 2.6; 2.7; 2.8; 2.9; 3; 3.1; 3.2; 3.3; 3.4; 3.5; 3.6; 3.7; 3.8; 3.9; 4); 4 cm or less (e.g., 3.9; 3.8; 3.7; 3.6; 3.5; 3.4; 3.3; 3.2; 3.1; 3; 2.9; 2.8; 2.7; 2.6; 2.5; 2.4; 2.3; 2.2; 2.1; 2; 1.9; 1.8; 1.7; 1.6; 1.5; 1.4; 1.3; 1.2; 1.1; 1); or 1 cm-4 cm (e.g., 1.1-4; 1.2-3.9; 1.3-3.8; 1.4-3.7; 1.5-3.6; 1.6-3.5; 1.7-3.4; 1.8-3.3; 1.9-3.2; 2-3.1; 2.1-3; 2.2-2.9; 2.3-2.8; 2.4-2.7; 2.5-2.6). Other aspects of the disclosure can be directed to the attachment region of the first end of the symmetrical arcuate portion comprising at least one aperture, such as but not limited to a keyhole, slotted hole, opening, or similar female receiving aperture. In some examples, the attachment region at the first end of the symmetrical arcuate portion (Fa) can comprise at least one projection, such as but not limited to a stud, screw, pin, elongated projection, or similar male inserting projection. Some embodiments provide for an attachment region of the disclosure, where the attachment region is configured for attachment to a handle.

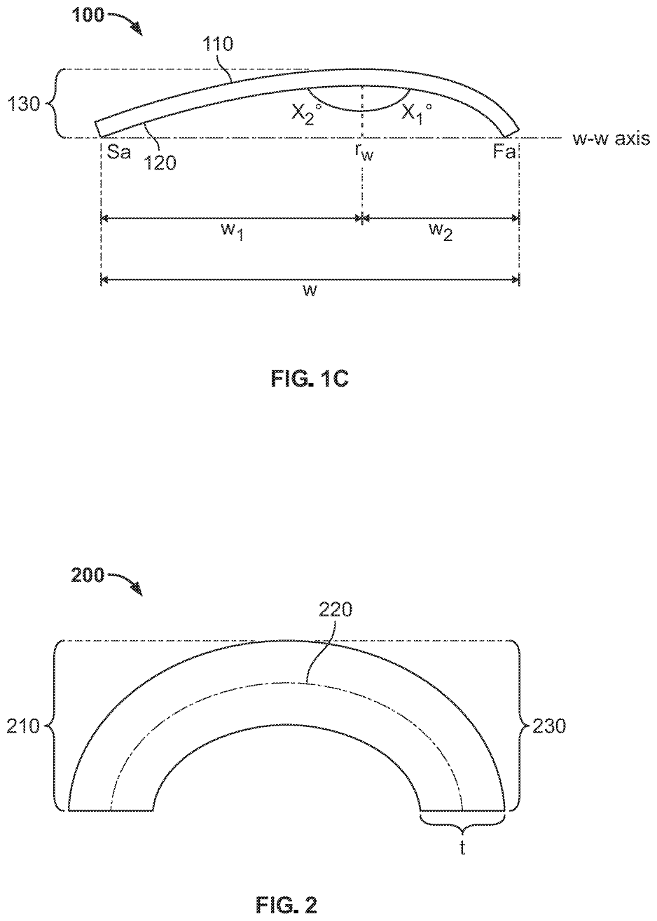

FIG. 2 shows a top view of some aspects of the medical blade of the disclosure, where the symmetrical arcuate portion is configured to taper from the top edge to the bottom edge, where the top edge comprises a thickness (“t”) that is thicker than a thickness of the bottom edge (dashed semi-circle line). For example, the top edge can have a thickness of about 0.4 mm, which tapers down to the bottom edge having a thickness of about 0.1 mm. In some embodiments, the thickness of the medical blade is constant or does not change from the top edge to the bottom edge. The thickness comprises a constant width selected from a range of 0.1 mm-0.4 mm. Other examples can be directed to medical blades of the description, where the symmetrical arcuate portion is configured to taper from the center of the top edge of the arc down to the first end and the second end of the symmetrical arcuate portion, such that the top edge tapers down and meets the bottom edge of the symmetrical arcuate portion of the cutting portion. In some embodiments, the medical blade of the disclosure comprises a symmetrical arcuate portion, where the symmetrical arcuate portion comprises the same thickness from the top edge down to the bottom edge, or the symmetrical arcuate portion does not taper from the top edge down to the bottom edge, rather maintains the same height throughout the symmetrical arcuate portion. In other examples, the medical blade of the description can comprise the symmetrical arcuate portion that tapers from the center of the top edge of the arc down to one end of the symmetrical arcuate portion (e.g. Fa or Sa). Some aspects provide for a medical blade, where the second end of the symmetrical arcuate portion comprises a tip or pointed end.

In some embodiments, the medical blade of the disclosure can be composed of a material selected from the group consisting of: metal (e.g., stainless steel (surgical grade or high grade stainless steel), high carbon steel, tempered steel), ceramic (e.g., zirconium, zirconium dioxide), titanium, diamond, sapphire, obsidian, combinations of metals, and metal ceramic alloys (e.g., cermet). The medical blades described here are sterile and can be disposable or single use medical blades, or alternatively, the medical blades are multi-use and sterilizable. In some examples, the medical blade of the disclosure is composed of a metal. Further examples provide for sterile disposable medical blades of the disclosure that are vacuum sealed and individually wrapped to ensure sterility and in order to avoid accidental punctures. The disposable medical blades can be sterilized by, for example, gamma radiation, autoclaving, or alcohol, and are rust-proof.

Various embodiments of the disclosure provide a medical blade having different dimensions of the symmetrical arcuate portion of, including but not limited to, a width, a rise, a height, a thickness between the concave side and the convex side of the cutting portion, an arc length of the symmetrical arcuate portion, and area encompassed by the symmetrical arcuate portion to the width axis (w-w axis). Some aspects of the disclosure provide a medical blade, where the symmetrical arcuate portion comprises a width of 0.5 mm or greater (e.g., 0.7, 0.9, 1.1, 1.3, 1.5, 1.7, 1.9, 3; 4; 5; 10; 20; 30; 40; 50). In some examples, the disclosure is directed to a medical blade, where the symmetrical arcuate portion comprises a width of 50 mm or less (e.g., 45; 35; 25; 15; 5.5; 4.5; 3.5; 2.5; 2; 1.8; 1.6; 1.4; 1.2; 1; 0.8; 0.6; 0.4). Some embodiments are directed to medical blades described here, where the symmetrical arcuate portion comprises a width of 0.5 mm-50 mm (e.g., 0.75-47.5; 1.25-44.5; 1.75-41.5; 2.25-38.5; 2.75-35.5; 3.25-32.5; 3.75-29.5; 4.25-26.5; 4.75-23.5; 5.25-20.5; 5.75-17.5; 6.25-14.5; 6.75-11.5; 7.25-8.5). In some examples, the disclosure is directed to a medical blade, where the symmetrical arcuate portion comprises a rise of 0.5 mm or greater (e.g., 0.7; 0.9; 1.1; 1.3; 1.5; 1.7; 1.9; 3; 4; 5; 10; 20; 30; 40; 50). Some aspects of the disclosure provide medical blades described here, where the symmetrical arcuate portion comprises a rise of 50 mm or less (e.g., 45; 35; 25; 15; 5.5; 4.5; 3.5; 2.5; 2; 1.8; 1.6; 1.4; 1.2; 1; 0.8; 0.6; 0.4). In yet some embodiments, the medical blades of the description comprising a symmetrical arcuate portion can comprise a rise of 0.5 mm-50 mm (e.g., 0.75-47.5; 1.25-44.5; 1.75-41.5; 2.25-38.5; 2.75-35.5; 3.25-32.5; 3.75-29.5; 4.25-26.5; 4.75-23.5; 5.25-20.5; 5.75-17.5; 6.25-14.5; 6.75-11.5; 7.25-8.5). Some aspects of the disclosure provide a medical blade, where the symmetrical arcuate portion comprises a height of 0.1 mm or greater (e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2). In some examples, the disclosure is directed to a medical blade, where the symmetrical arcuate portion comprises a height of 2 mm or less (e.g., 1.95, 1.85, 1.75, 1.65, 1.55, 1.45, 1.35, 1.25, 1.15, 1.05, 0.95, 0.85, 0.75, 0.65, 0.55, 0.45, 0.35, 0.25, 0.15). Some embodiments are directed to medical blades described here, where the symmetrical arcuate portion comprises a height of 0.1 mm-2 mm (e.g., 0.2-1.92; 0.3-1.82; 0.4-1.72; 0.5-1.62; 0.6-1.52; 0.7-1.42; 0.8-1.32; 0.9-1.22; 1-1.12; 1 mm). In some aspects, the medical blades described here comprising the symmetrical arcuate portion having a thickness between the concave side and the convex side of the cutting portion, of 0.01 mm or greater (e.g., 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71. 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2). Some examples provide for medical blades described here, where the symmetrical arcuate portion comprises a thickness between the concave side and the convex side of the cutting portion of 2 mm or less (e.g., 2, 1.9, 1.8, 1.7, 1.6, 1.5, 1.4, 1.3, 1.2, 1.1, 1, 0.99, 0.98, 0.97, 0.96, 0.95, 0.94, 0.93, 0.92, 0.91, 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.32, 0.31, 0.3, 0.29, 0.28, 0.27, 0.26, 0.25, 0.24, 0.23, 0.22, 0.21, 0.2, 0.19, 0.18, 0.02, 0.03, 0.04, 0.05, 0.06, 0.07, 0.08, 0.09, 0.1, 0.11, 0.12, 0.13, 0.14, 0.15, 0.16, 0.17, 0.16, 0.02, 0.03, 0.04, 0.05, 0.06, 0.07, 0.08, 0.09, 0.1, 0.11, 0.12, 0.13, 0.14, 0.15, 0.15, 0.14, 0.13, 0.12, 0.11, 0.1, 0.09, 0.08, 0.07, 0.06, 0.05, 0.04, 0.03, 0.02, 0.01). In some embodiments, the medical blades described here comprise a symmetrical arcuate portion having a thickness between the concave side and the convex side of the cutting portion of 0.01 mm-2 mm (e.g., 0.02-1.9, 0.03-1.8, 0.04-1.7, 0.05-1.6, 0.06-1.5, 0.07-1.4, 0.08-1.3, 0.09-1.2, 0.1-1.1, 0.11-1, 0.12-0.99, 0.13-0.98, 0.14-0.97, 0.15-0.96, 0.16-0.95, 0.17-0.94, 0.18-0.93, 0.19-0.92, 0.2-0.91, 0.21-0.9, 0.22-0.89, 0.23-0.88, 0.24-0.87, 0.25-0.86, 0.26-0.85, 0.27-0.84, 0.28-0.83, 0.29-0.82, 0.3-0.81, 0.31-0.8, 0.32-0.79, 0.33-0.78, 0.34-0.77, 0.35-0.76, 0.36-0.75, 0.37-0.74, 0.38-0.73, 0.39-0.72, 0.4-0.71, 0.41-0.7, 0.42-0.69, 0.43-0.68, 0.44-0.67, 0.45-0.66, 0.46-0.65, 0.47-0.64, 0.48-0.63, 0.49-0.62, 0.5-0.61, 0.51-0.6, 0.52-0.59, 0.53-0.58, 0.54-0.57, 0.55-0.56). Some examples provide for medical blades described here, where the length of the arc of the symmetrical arcuate portion comprises 1.4 mm or greater (e.g., 1.75, 2.25, 2.75, 3.25, 3.75, 4.25, 4.75, 5.25, 5.75, 6.25, 6.75, 7.25, 7.75, 8.25, 8.75, 9.25, 9.75, 10.25, 10.75, 15.5, 22, 27, 32, 37, 42, 47, 52, 57, 62, 67, 72, 77, 82, 87, 92, 97, 102, 107, 112, 117, 122, 127, 132, 137). In some embodiments, the medical blades described here comprise a length of the arc of the symmetrical arcuate portion of 140 or less (e.g., 138, 134, 128, 124, 118, 114, 108, 104, 98, 94, 88, 84, 78, 74, 68, 64, 58, 54, 48, 44, 38, 34, 28, 24, 18, 14, 8, 4, 2.8, 2.4, 1.8, 1.4). Some aspects provide for medical blades described here, where the length of the arc of the symmetrical arcuate portion ranges from 1.4 mm to 139 mm (e.g., 1.5-135, 2-130, 3-125, 4-120, 5-115, 6-110, 7-105, 8-100, 9-95, 10-90, 11-85, 12-80, 13-75, 14-70, 15-65, 20-60, 25-55, 30-50, 35-45). In some examples, the medical blades described here comprise an area of the symmetrical arcuate portion to the width axis or w-w axis of 0.26 mm² or greater (e.g., 0.36, 0.46, 0.56, 0.66, 0.76, 0.86, 0.96, 1.6, 2.6, 3.6, 4.6, 5.6, 6.6, 7.6, 8.6, 9.6, 10.6, 11.6, 12.6, 13.6, 14.6, 15.6, 26, 36, 46, 56, 66, 76, 86, 96, 106, 116, 126, 136, 146, 156, 166, 176, 186, 196, 206, 216, 226, 236, 246, 256). Some aspects provide for medical blades described here, where the area of the symmetrical arcuate portion to the width axis or w-w axis comprises 263 mm² or less (e.g., 260, 250, 240, 230, 220, 210, 200, 190, 180, 170, 160, 150, 140, 130, 120, 110, 100, 90, 80, 70, 60, 50, 40, 30, 20, 10, 5, 4, 3, 2, 1, 0.5, 0.4, 0.3). In some embodiments, the medical blades described here comprise an area of the symmetrical arcuate portion to the width axis or w-w axis ranging from 0.26 mm²-263 mm² (e.g., 0.3-260, 0.5-255, 1-250, 1.5-245, 2-240, 2.5-235, 3-230, 3.5-225, 4-220, 2.5-215, 3-210, 3.5-200, 4-295, 4.5-290, 5-285, 5.5-280, 6-275, 6.5-270, 7-265, 7.5-260, 8-255, 8.5-250, 9-245, 9.5-240, 10-235, 15-230, 20-225, 25-220, 30-215, 35-210, 40-205, 45-200, 50-195, 55-190, 60-185, 65-180, 70-175, 75-170, 80-165, 85-160, 90-155, 95-150, 100-145, 105-140, 110-135, 115-130, 120-125). A person of ordinary skill and/or a practitioner in the field of dermatology, surgery, pathology, or any field that requires the removal and/or use of a skin sample or skin biopsy has knowledge and an understanding of dimensions of the medical blade useful for obtaining such skin samples or skin biopsies.

Some examples provide for a medical blade comprising a symmetrical arcuate portion comprising a semi-circle with a width greater than a rise; or a width of 1 cm and a rise of 0.5 cm, where the semi-circular symmetrical arcuate portion comprises a height of the symmetrical arcuate portion of 0.1 mm-5 cm. Non-limiting examples of a medical blade described here include those comprising a symmetrical arcuate portion comprising a semi-elliptical shape with a height of 0.1 cm-1 cm and: a width of 3 cm and a rise of 0.2 cm; a width of 2 cm and a rise of 0.2 cm; or a width of 1 cm and a rise of 0.2 cm. In yet other examples, a medical blade of the description can comprise a symmetrical arcuate portion comprising a height of 0.1 cm-1 cm and: a width of 1 cm and a rise of 1 cm; a width of 1 cm and a rise of 0.2 cm; a width of 0.6 cm and a rise of 0.6 cm; a width of 0.6 cm and a rise of 0.3 cm; a width of 0.6 cm and a rise of 0.2 cm; a width of 0.2 cm and a rise of 0.2 cm; or any combinations thereof. In some embodiments, a medical blade of the disclosure comprises a width of about 2 cm, a rise of about 5 mm, a height of about 5 mm to about 10 mm, and a thickness of about 0.1 mm. Further embodiments provide for a medical blade of the disclosure comprising a width of about 2 cm, a rise of about 5 mm, a height of about 5 mm, and a thickness of about 0.1 mm. In some embodiments, a medical blade of the disclosure comprises a J shape or a question mark shape.

### Body Portion

In some embodiments of the medical blade described here, the medical blade can comprise a cutting portion, having, for example, a symmetrical arcuate portion, and a body portion, where the body portion comprises an attachment region configured for fixed attachment to a handle. Some aspects of the disclosure provide for the first end of the body portion to be fixedly attached to the second end of the handle, where the attachment can be temporary, permanent, non-folding, non-sliding, and/or secure.

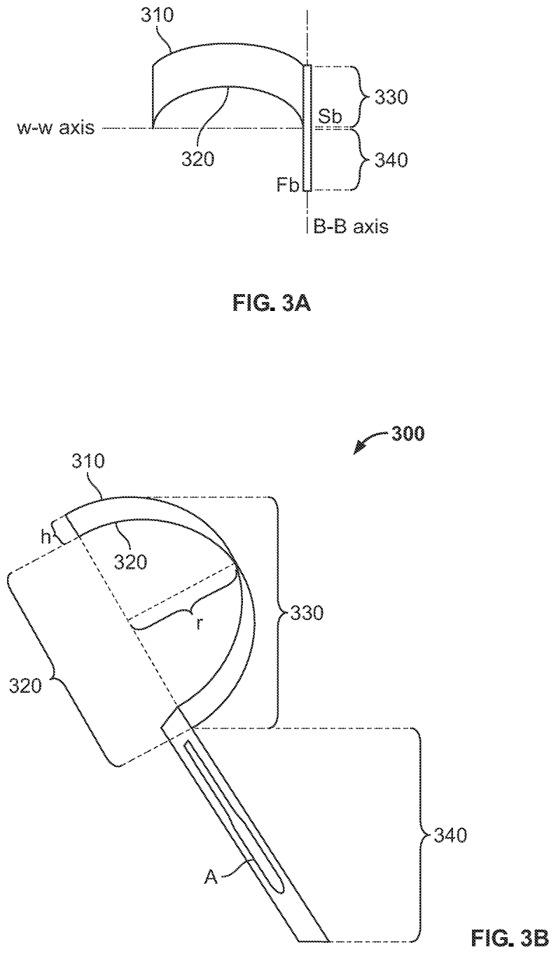

FIG. 3, in some embodiments, provides for a medical blade of the description, including for example, a surgical blade, as described here, comprising a symmetrical arcuate portion comprising a first end of the symmetrical arcuate portion (Fa), a second end of the symmetrical arcuate portion (Sa), and an arc therebetween, where the first end of the symmetrical arcuate portion (Fa) and the second end of the symmetrical arcuate portion (Sa) are on an axis (w-w axis), where the axis is the same w-w axis, and the second end of the symmetrical arcuate portion (Sa) is distal to the first end of the symmetrical arcuate portion (Fa), and further comprise a body portion that is fixedly attached at the first end (Fa) of the symmetrical arcuate portion and at the second end (Sb) of the body portion. The body portion can comprise a first end (Fb) or proximal end and a second end (Sb) or distal end, where the body portion is linear or essentially linear, extending along an axis (B-B), for example, a longitudinal axis, between the first end of the body portion (Fb) and the second end of the body portion (Sb). In some aspects, the second end of the body portion (Sb) is distal to the first end (Fb), and the cutting portion of the described medical blade is configured to be fixed at the first end of the symmetrical arcuate portion (Fa) and the second end of the body portion (Sb). Some examples provide for medical blades as described here, where the body portion is also configured to be fixed at the first end of the body portion (Fb). In some embodiments, the body portion of some medical blades as described here can be fixed, fixedly attached, or operably connected at the first end of the body portion (Fb) to a handle, such as a second end of a handle (Sh) distal to a first end of the handle (Fh). Exemplary medical blades described here comprise a cutting portion or a symmetrical arcuate portion (330) and a body portion (340), where the Fb is configured to fixedly attach to a handle via respective attachment regions. See, e.g., FIGS. 3A-3B.

Other aspects of the disclosure provide for medical blades comprising a symmetrical arcuate portion and a body portion, where the first end of the symmetrical arcuate portion (Fa) is fixedly attached to the second end of the body portion (Sb), and first end of the body portion (Fb) can be operably connected, fixedly attached, or fixed to a second end of a handle (Sh), which is distal to a first end of a handle (Fh), where the first end of the body portion (Fb) comprises an attachment region. Some examples described here provide an attachment region comprising at least one aperture, such as but not limited to a keyhole, a slotted hole, an opening, or a similar female receiving aperture. In some embodiments, the attachment region at the first end of the symmetrical arcuate portion (Fa) can comprise at least one projection, such as but not limited to a stud, a screw, a pin, an elongated projection, or a similar male inserting projection. Some aspects provide for an attachment region of the disclosure, where the attachment region of the medical blade (e.g., symmetric arcuate portion or body portion) is configured for attachment to a handle, such as at the second end of the handle (Sh), which is distal to the first end of the handle (Fh), by fixedly attaching or locking the male projection and the female receiving aperture that are aligned on their respective medical blade and handle.

An exemplary medical blade comprises (a) a cutting portion (e.g., symmetrical arcuate portion) comprising a cutting edge, where the cutting edge is the bottom edge or the top edge, or both the bottom edge and the top edge, where the rise (r) is equivalent to or essentially similar to the length that is half the width (w) (e.g., r=1 cm, w=2 cm); and (b) a body portion (e.g., 2.5 cm length) comprising an attachment region, for example a female receiving aperature, where the female receiving aperature comprises a wide portion (e.g., 1 cm×0.3 cm) and a narrow portion (e.g., 0.8 cm×0.15 cm) that forms a slotted hole or opening, where the body portion is attached to the cutting portion at a 90° angle or about a 90° angle or greater (e.g., 90°-180°; 90°-135°), such that the attachment region of the medical blade fixedly attaches to a handle comprising an attachment region comprising a male inserting projection that is aligned to the female receiving aperture, securely locking or fixedly attaching the medical blade to the handle by sliding the male inserting projection end of the handle into (or to unlock or separate, out of) the female receiving aperture (e.g., key slot). See, e.g., FIGS. 3A-3B.

### Scalpel

### Handle

In some embodiments, the medical blades described here can be used in conjunction with a handle to form a scalpel. Handles of the description are generally linear, including but not limited to elongated, essentially linear, or along a longitudinal axis, comprising a first end of the handle and a second end of the handle, where the first end is a proximal end and the second end is distal to the first end. Handles of the disclosure can attach, be fixedly attached, or operably fixed to medical blades described here. For example, the second end of the handle is configured to attach to a first end of the symmetrical arcuate portion of the cutting portion or to a first end of the body portion of the medical blade described here. In some aspects, the medical blade can comprise a semi-circular arcuate portion fixedly attached to a linear body portion forming or configured to form a question mark shape, where the first end of the body portion is fixedly attached to a second end of a handle.

Any type of handle that can securely attach to the medical blade can be used. Non-limiting examples of handles include: a flat handle, a cylindrical handle (e.g., resembling a pen), an ergonomic handle, a plastic handle, a disposable handle, a reusable handle, a stainless steel handle, a titanium handle, a handle with grips, and any combinations thereof. In some examples, the scalpel of the disclosure is sterile or capable of being sterilized; disposable or configured for one time use; or for multiple uses and sterilizable by, for example, gamma radiation, autoclaving, alcohol.

In some embodiments, the scalpel of the disclosure can comprise at least one projection at the second end of the handle. Non-limiting examples of the at least one projection at the second end of the handle include a stud, a screw, a pin, an elongated projection, a similar male inserting projection, or any combinations thereof. Some examples of handles of the disclosure include at least one aperture selected from the group consisting of: a keyhole, a slotted hole, an opening, a similar female receiving aperture, and any combinations thereof. The described scalpels comprise a medical blade having at least one projection or at least one aperture and a handle having at least one projection or at least one aperture for fixedly attaching the medical blade and the handle, where the medical blade and the handle have different means for attachment, i.e., the medical blade and the handle do not comprise the same attachment means. In some examples, the scalpel of the disclosure comprises a medical blade comprising at least one projection and a handle comprising at least one aperture, where the scalpel is configured by attachment of the at least one projection of the medical blade and the at least one aperture of the handle. Other examples provide scalpels of the disclosure comprising a medical blade comprising at least one aperture and a handle comprising at least one projection, where the scalpel is configured by attachment of the at least one aperture of the medical blade and the at least one projection of the handle. Embodiments of the disclosure provide for attaching regions or attachment regions of the medical blade and the handle, where the medical blade comprises at least one aperture, while the handle comprises at least one projection, or where the medical blade comprises at least one projection, while the handle comprises at least one aperture, where the at least one projection and the at least one aperture are aligned in a manner such that the medical blade and the handle are configured to be fixedly attached or locked to form a scalpel comprising the medical blade and the handle. Other exemplary embodiments are directed to the attaching regions of the medical blade, which comprises at least one aperture and at least one projection which are each aligned with the opposing attaching regions of the handle that comprises at least one projection and at least one aperture, such that the medical blade and the handle are configured to be fixedly attached or locked to form a scalpel comprising the medical blade and the handle. In some examples, the scalpel of the disclosure does not comprise a medical blade comprising at least one aperture and a handle comprising at least one aperture, where each of the at least one apertures is aligned such that the medical blade cannot be fixedly attached to the handle. Further examples provide the scalpel of the disclosure that does not comprise a medical blade comprising at least one projection and a handle comprising at least one projection, where each of the at least one projections is aligned such that the medical blade cannot be fixedly attached to the handle. Some aspects of the disclosure provide scalpels described here, where the second end of the handle comprises at least one projection (e.g., male inserting projection: stud, screw, pin, elongated projection) that is configured to fasten or securely fasten in the at least one aperture (e.g., female receiving aperture: keyhole, slotted hole, opening) of the medical blade, where the male inserting projection is aligned with the female receiving aperture to form a medical blade fixedly attached to a handle. In other examples, scalpels of the disclosure comprising a medical blade and a handle, where the second end of the handle comprises at least one aperture (e.g., female receiving aperture: keyhole, slotted hole, opening) that is configured to fasten or securely fasten in the at least one projection (e.g., male inserting projection: stud, screw, pin, elongated projection) of the medical blade.

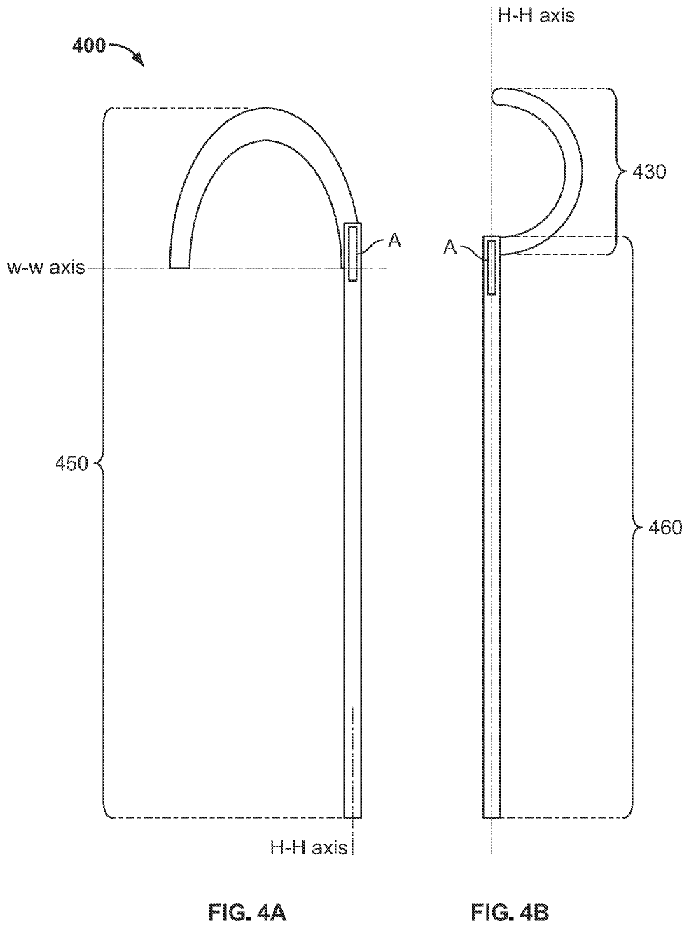

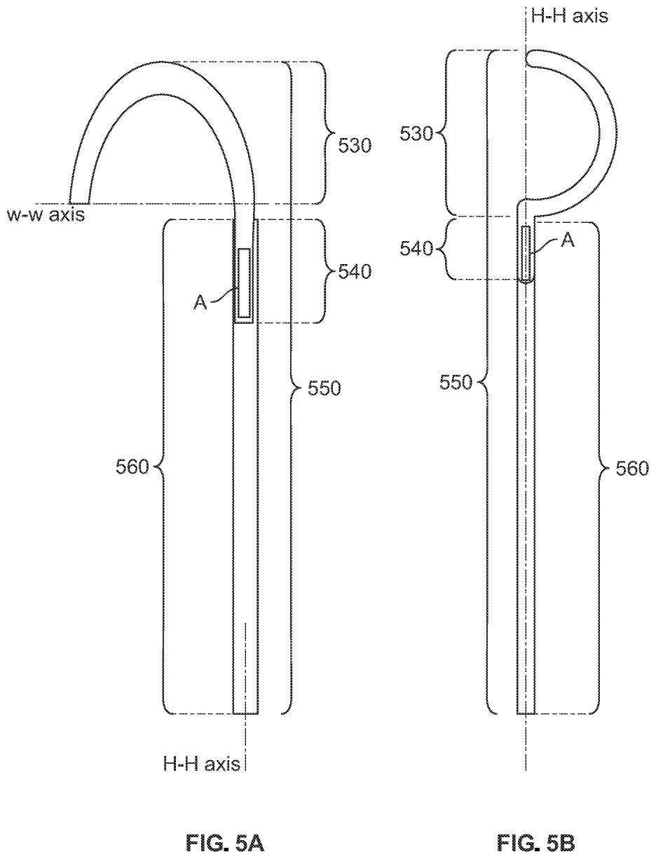

FIGS. 4A-4B and FIGS. 5A-5B show various embodiments of exemplary scalpels of the disclosure. In FIG. 4A, the scalpel comprises a medical blade comprising a symmetric arcuate portion, where the first end of the symmetrical arcuate portion (Fa) and the second end of the symmetrical arcuate portion (Sa) extend along the w-w axis, which forms a width of the symmetric arcuate portion therebetween; and a handle comprising a first end of the handle (Fh) and a second end of the handle (Sh) extending along the H-H axis, where the w-w axis along which the width of the symmetric arcuate portion lies, is perpendicular, essentially perpendicular, or substantially perpendicular (e.g., ˜80°-˜100°, ˜85°, 90°, or 95° from the H-H axis or longitudinal axis) to the H-H axis, and the symmetric arcuate portion and the handle are fixedly attached at an attaching region (A) located at the first end of the symmetric arcuate portion (Fa) and the second end of the handle (Sh). FIG. 4B shows an exemplary scalpel, where the first end (Fa) and the second end (Sa) of the symmetric arcuate portion extend along the H-H axis, which is the same H-H axis as that of the handle, which extends between the first end (Fh) and the second end (Sh) of the handle, where the scalpel comprises a medical blade comprising the symmetric arcuate portion and the handle are fixedly attached at an attaching region (A) located at the first end of the symmetric arcuate portion (Fa) and the second end of the handle (Sh). Some aspects of the disclosure comprise a scalpel as described here, where the scalpel comprises any of the medical blades described here and any of the handles described here, where the handle is linear, elongated, or essentially linear along a longitudinal axis or H-H axis, where the handle comprises a first end of the handle (Fh) or proximal end and a second end of the handle (Sh) or distal end, where the second end of the handle (Sh) is fixedly attached, temporarily attached, or permanently attached, where the attachment is secure, non-folding, or non-sliding, where the second end of the handle (Sh) and the attachment region of the medical blade are securely fastened or where the second end of the handle (Sh) is fixedly attached at the attachment region of the medical blade.

In FIGS. 5A-5B, the exemplary scalpel comprising a medical blade described here and a handle, where the medical blade comprises a symmetric arcuate portion and a body portion, where the symmetric arcuate portion and the body portion are fixedly attached at the first end of the symmetric arcuate portion (Fa) and the second end of the body portion (Sb), and the first end of the body portion (Fb) and the second end of the handle (Sh) are configured to attach at the attaching region (A). In the exemplary scalpel of FIG. 5A, the scalpel comprises a medical blade comprising: a symmetric arcuate portion, where the first end of the symmetrical arcuate portion (Fa) and the second end of the symmetrical arcuate portion (Sa) extend along the w-w axis, which forms a width of the symmetric arcuate portion therebetween; and a body portion, where the second end of the body portion (Sb) is fixedly connected or attached to the first end of the symmetric arcuate portion; and the scalpel further comprises a handle comprising a first end of the handle (Fh) and a second end of the handle (Sh) distal from the first end of the handle (Fh), where the handle extends along the H-H axis, where the w-w axis along which the width of the symmetric arcuate portion lies, is perpendicular, essentially perpendicular, or substantially perpendicular (e.g., ˜80°-˜100° from the H-H axis or longitudinal axis) to the H-H axis, and the symmetric arcuate portion and the handle are fixedly attached at an attaching region (A) located at the first end of the symmetric arcuate portion (Fa) and the second end of the handle (Sh). FIG. 5B shows an exemplary scalpel, where the scalpel comprises a medical blade comprising: a symmetric arcuate portion, where the first end of the symmetrical arcuate portion (Fa) and the second end of the symmetrical arcuate portion (Sa) extend along the w-w axis, which forms a width of the symmetric arcuate portion therebetween; and a body portion, where the second end of the body portion (Sb) is fixedly connected or attached to the first end of the symmetric arcuate portion; and the scalpel further comprises a handle comprising a first end of the handle (Fh) and a second end of the handle (Sh) distal from the first end of the handle (Fh), where the handle extends along the H-H axis, where the axis along which the width of the symmetric arcuate portion lies, is the same as that of the handle or the H-H axis, and the symmetric arcuate portion of the medical blade and the handle are fixedly attached at an attaching region (A) located at the first end of the symmetric arcuate portion (Fa) and the second end of the handle (Sh). Some aspects of the disclosure comprise a scalpel as described here, where the scalpel comprises any of the medical blades described here and any of the handles described here, where the handle is linear, elongated, or essentially linear along a longitudinal axis or H-H axis, where the handle comprises a first end of the handle (Fh) or proximal end and a second end of the handle (Sh) or distal end, where the second end of the handle (Sh) is fixedly attached, temporarily attached, or permanently attached, where the attachment is secure, non-folding, or non-sliding, where the second end of the handle (Sh) and the attachment region of the medical blade are securely fastened or where the second end of the handle (Sh) is fixedly attached at the attachment region of the medical blade.

### Uses and Methods of Using the Medical Blades and Scalpels of the Description

In various embodiments of the disclosure, the medical blades and scalpels as described here, can be used in methods for obtaining a skin sample from a subject. As used here, the term “subject” refers to any organism to which a medical blade or a scalpel (both of which are sterile) comprising the medical blade as described here can be applied, e.g., for experimental, diagnostic, prophylactic, and/or therapeutic purposes. Typical subjects include any animal (e.g., mammals such as mice, rats, rabbits, cats, dogs, goats, pigs, horses, cattle, non-human primates, and humans). A subject in need thereof is typically a subject for whom it is desirable to obtain a skin sample or skin biopsy using any one of the cutting devices, including but not limited to the medical blades comprising a cutting portion alone or a cutting portion and a body portion, and/or the scalpels comprising a medical blade and a handle as described herein. For example, a subject in need thereof may seek or be in need of treatment, require treatment, be receiving treatment, may be receiving treatment in the future, or a human or animal who is under care of a trained professional for a particular disease, disorder, or condition, such as a skin disease, disorder, or condition, and the subject in need requires diagnosis, monitoring, or assessment of such a skin disease, disorder, or condition, where monitoring or assessment can be determined by comparing skin before and after treatment for the skin disease, disorder, or condition.

Some examples of the disclosure provide a use of any one of the medical blades described here or any one of the scalpels described here, for obtaining a skin sample, a skin biopsy, a tissue sample, or a tissue biopsy from a subject, where the subject is in need thereof. Also encompassed by the disclosure are uses of any of the medical blades described here for obtaining a skin sample, a skin biopsy, a tissue sample, or a tissue biopsy from a subject. In some aspects, the disclosure provides uses of any one of the scalpels described here for obtaining a skin sample, a skin biopsy, a tissue sample, or a tissue biopsy from a subject.

In some embodiments, any of the disclosed scalpels comprising any one of the described medical blades and any of the described handles can be used for obtaining a skin sample, a skin biopsy, a tissue sample, or a tissue biopsy from a subject who is in need of a diagnosis for a skin disease, condition, or disorder or from a subject who has been diagnosed as having a skin disease, condition, or disorder, where the subject is in need of monitoring the progression of or improvement upon treatment for a skin disease, condition, or disorder. Other examples provide for uses of the scalpels of the disclosure for diagnosing a skin disease, condition, or disorder by obtaining a skin sample, a skin biopsy, a tissue sample, or a tissue biopsy from a subject for analysis and/or diagnosis of the subject. Some aspects can be directed to uses of the described scalpels for removing abnormal tissue from a subject.

In some examples of the disclosure, methods of obtaining a skin sample, a skin biopsy, a tissue sample, or a tissue biopsy from a subject are provided, where the method comprises: moving the cutting portion described here, which includes moving the symmetrical arcuate portion, through the surface of skin or moving the cutting portion to scrape the surface or surface layer of skin of a subject, to obtain a skin sample, a skin biopsy, a tissue sample, or a tissue biopsy from the subject. The step of moving the cutting portion of the methods can occur around the longitudinal axis or axis of the handle (H-H axis), such that the moving comprises a downward motion through or across the surface of skin from the subject to obtain a skin sample. The methods of obtaining a skin sample described here can in some embodiments comprise moving the cutting portion of the description around an axis perpendicular to the longitudinal axis or perpendicular to the axis of the handle (H-H axis), or moving the cutting portion of the description around an axis of the width (w-w axis) of the symmetrical arcuate portion, such that the moving comprises a downward motion through or across the surface of skin from the subject to obtain a skin sample. In some aspects of the disclosure, the described methods can be directed to moving any of the cutting portions described here, where the moving a downward motion and an upward motion. Other aspects of the method described here provide for moving the cutting portion of any one of the scalpels disclosed here in a downward and an upward motion to remove or obtain a skin sample from the subject. Further examples of the methods of the disclosure include methods comprising moving the cutting portion or the cutting portion of the scalpel, where the moving comprises scraping the cutting portion across and through a portion of the skin surface of the subject to obtain a skin sample.

Some examples of the disclosure provide any of the methods of obtaining a skin sample (including, for example, skin biopsy, tissue sample, tissue biopsy) by moving the cutting portion of a medical blade or a scalpel described here, where the cutting portion comprises a symmetrical arcuate portion comprising a width and a rise that results in obtaining a skin sample having a specific diameter, where, when moved through the surface of skin of a subject, the symmetrical arcuate portion of a medical blade or a scalpel comprising: a width of 1 cm and a rise of 0.1 cm, results in obtaining a skin sample comprising a 0.6 cm diameter; a width of 1 cm and a rise of 0.2 cm, results in obtaining a skin sample comprising a 0.8 cm diameter; a width of 1 cm and a rise of 0.3 cm, results in obtaining a skin sample comprising a 0.92 cm diameter; a width of 1 cm and a rise of 0.4 cm, results in obtaining a skin sample comprising a 0.98 cm diameter; a width of 1 cm and a rise of 0.5 cm, results in obtaining a skin sample comprising a 1 cm diameter; a width of 0.6 cm and a rise of 0.1 cm, results in obtaining a skin sample comprising a 0.45 cm diameter; a width of 0.6 cm and a rise of 0.2 cm, results in obtaining a skin sample comprising a 0.57 cm diameter; a width of 0.6 cm and a rise of 0.3 cm, results in obtaining a skin sample comprising a 0.6 cm diameter; a width of 0.5 cm and a rise of 0.1 cm, results in obtaining a skin sample comprising a 0.4 cm diameter; a width of 0.5 cm and a rise of 0.2 cm, results in obtaining a skin sample comprising a 0.49 cm diameter.

Provided here are some aspects of the disclosure, which are directed to methods of removing a skin sample from a subject, comprising: moving a cutting portion of a scalpel through skin of a subject in a downward motion around an axis in a manner to cutout, scoop out (i.e., downward motion followed by an upward motion), scrape skin of the subject, or combinations thereof. In some embodiments, the methods of removing a skin sample from a subject comprise moving a cutting portion of a scalpel through skin of a subject in a downward motion around an axis, perpendicular to the surface of the skin, in a manner to cut down and then in an upward motion around the axis through the surface of the skin to obtain a skin sample. The scalpel useful in removing a skin sample from a subject comprises a medical blade and a scalpel, where the medical blade comprising a cutting portion, where the cutting portion comprises a symmetrical arcuate portion, where the symmetrical arcuate portion comprises a first end of the symmetrical arcuate portion, a second end of the symmetrical arcuate portion, and an arc therebetween, where the first end of the symmetrical arcuate portion and the second end of the symmetrical arcuate portion are each on an axis, and the axis is the same axis. The second end of the symmetrical arcuate portion is distal to the first end of the symmetrical arcuate portion, where a width extends on the axis between the first end of the symmetrical arcuate portion and the second end of the symmetrical arcuate portion. The symmetrical arcuate portion comprising an arc between the first end of the symmetrical arcuate portion and the second end of the symmetrical arcuate portion, where the arc comprises a rise extending from the center of the width to the center of the arc. The symmetrical arcuate portion of the scalpel comprises: (i) a convex side; (ii) a concave side; (iii) a top edge; and (iv) a bottom edge, where the bottom edge comprises a cutting edge, and comprises a height extending between the top edge and the bottom edge of the symmetrical arcuate portion. The cutting portion of the medical blade useful in such methods, is configured to be fixed (or fixedly configured, operatively connectable) at the first end of the symmetrical arcuate portion (see, e.g., FIG. 4A-FIG. 4B) or, if the medical blade comprises a cutting portion fixed (or fixedly configured, operatively connectable) to a body portion, then the body portion of the medical blade is configured to be fixed (or fixedly configured, operatively connectable) at the first end of the body portion (see, e.g., FIG. 5A-FIG. 5B), where the first end of the symmetrical arcuate portion or the first end of the body portion are fixed (or fixedly configured, operatively connectable) to a second end of a handle of the scalpel. Methods of using the scalpel described here comprise moving or rotating the scalpel around an axis along the axis of the width (w-w axis), around an axis perpendicular to a longitudinal axis or H-H axis of the handle, or towards the first end or proximal end of the handle (see, FIG. 4A, FIG. 5A) or methods of using the scalpel described here comprising moving or rotating the scalpel around a longitudinal axis or H-H axis of the handle (see, FIG. 4B, FIG. 5B), to remove a skin sample from a subject in need thereof. The subject in need thereof includes a subject in need of a diagnosis of a disease, condition, or disorder; a subject in need of monitoring progression of a disease, condition, or disorder; a subject in need of removing abnormal skin, where the abnormal skin can be skin having a disease, a condition, or a disorder or where the skin is not healthy free of any disease, condition, or disorder.

In some embodiments, methods of using the scalpel of the disclosure results in obtaining clean skin samples with sharp borders, while requiring less manipulation of the scalpel. The dimensions and the configuration of the cutting portion of the cutting devices of the disclosure, including the medical blades and the scalpels, allow and enable obtaining and removing various skin samples, skin biopsies, tissue samples, or tissue biopsies having varying depths and diameters sufficient to obtain and remove whole samples or biopsies in a single, fluid motion, which advantageously results in clean edged and unadulterated samples or biopsies.

### Specific Embodiments

Non-limiting specific embodiments are described below each of which is considered to be within the present disclosure.

Embodiment 1. A medical blade (e.g., surgical), comprising: a cutting portion, wherein the cutting portion comprises a symmetrical arcuate portion, wherein the symmetrical arcuate portion comprises a first end of the symmetrical arcuate portion, a second end of the symmetrical arcuate portion, and an arc therebetween, wherein the first end of the symmetrical arcuate portion and the second end of the symmetrical arcuate portion are on an axis, wherein the axis is the same axis, wherein the second end of the symmetrical arcuate portion is distal to the first end of the symmetrical arcuate portion, wherein the symmetrical arcuate portion comprises a width extending on the axis between the first end of the symmetrical arcuate portion and the second end of the symmetrical arcuate portion, wherein the arc comprises a rise extending from the center of the width to the center of the arc, wherein the symmetrical arcuate portion comprises: (i) a convex side; (ii) a concave side; (iii) a top edge; and (iv) a bottom edge, wherein the bottom edge comprises a cutting edge, wherein the symmetrical arcuate portion comprises a height extending between the top edge and the bottom edge, wherein the cutting portion is configured to be fixed (or fixedly configured, operatively connectable) at the first end of the symmetrical arcuate portion.

Embodiment 2. The medical blade (e.g., surgical) of embodiment 1, further comprising: a body portion, wherein the body portion comprises a first end (proximal) and a second end (distal), wherein the body portion is linear (e.g., essentially linear), extending along a longitudinal axis between the first end of the body portion and the second end of the body portion, wherein the second end of the body portion is distal to the first end of the body portion, wherein the cutting portion is configured to be fixed at the second end of the body portion, wherein the body portion is configured to be fixed at the first end of the body portion.

Embodiment 3. The medical blade of embodiment 1 or embodiment 2, wherein the medical blade is a surgical blade.

Embodiment 4. The medical blade of any one of the preceding embodiments, wherein the medical blade comprises a shape is selected from the group consisting of: a parabolic shape, a semi-elliptical shape, a semi-circular shape, a semi-ovoid shape, a C-shape, a J-shape, and a question mark shape.

Embodiment 5. The medical blade of any one of the preceding embodiments, wherein the axis of the width extending between the first end of the symmetrical arcuate portion and the second end of the symmetrical arcuate portion is a longitudinal axis.

Embodiment 6. The medical blade of any one of embodiments 1-4, wherein the axis of the width extending between the first end of the symmetrical arcuate portion and the second end of the symmetrical arcuate portion is a perpendicular to the longitudinal axis.

Embodiment 7. The medical blade of embodiment 1, wherein the first end of the symmetrical arcuate portion comprises an attachment region.

Embodiment 8. The medical blade of embodiment 2, wherein the first end of the body portion comprises an attachment region.

Embodiment 9. The medical blade of embodiment 7 or embodiment 8, wherein the attachment region comprises at least one aperture (e.g., keyhole, slotted hole, opening).

Embodiment 10. The medical blade of any one of the preceding embodiments, wherein the symmetrical arcuate portion is configured to taper from the top edge to the bottom edge.

Embodiment 11. The medical blade of any one of the preceding embodiments, wherein the second end of the symmetrical arcuate portion comprises a tip.

Embodiment 12. The medical blade of embodiment 9, wherein the attachment region is configured for attachment to a handle.

Embodiment 13. The medical blade of any one of the preceding embodiments, wherein the width comprises 0.5 mm or greater (e.g., 0.7, 0.9, 1.1, 1.3, 1.5, 1.7, 1.9, 3; 4; 5; 10; 20; 30; 40; 50); 50 mm or less (e.g., 45; 35; 25; 15; 5.5; 4.5; 3.5; 2.5; 2; 1.8; 1.6; 1.4; 1.2; 1; 0.8; 0.6; 0.4); or 0.5 mm-50 mm (e.g., 0.75-47.5; 1.25-44.5; 1.75-41.5; 2.25-38.5; 2.75-35.5; 3.25-32.5; 3.75-29.5; 4.25-26.5; 4.75-23.5; 5.25-20.5; 5.75-17.5; 6.25-14.5; 6.75-11.5; 7.25-8.5).

Embodiment 14. The medical blade of any one of the preceding embodiments, wherein the rise comprises 0.5 mm or greater (e.g., 0.7; 0.9; 1.1; 1.3; 1.5; 1.7; 1.9; 3; 4; 5; 10; 20; 30; 40; 50); 50 mm or less (e.g., 45; 35; 25; 15; 5.5; 4.5; 3.5; 2.5; 2; 1.8; 1.6; 1.4; 1.2; 1; 0.8; 0.6; 0.4); or 0.5 mm-50 mm (e.g., 0.75-47.5; 1.25-44.5; 1.75-41.5; 2.25-38.5; 2.75-35.5; 3.25-32.5; 3.75-29.5; 4.25-26.5; 4.75-23.5; 5.25-20.5; 5.75-17.5; 6.25-14.5; 6.75-11.5; 7.25-8.5).

Embodiment 15. The medical blade of any one of the preceding embodiments, wherein the height comprises 0.1 mm or greater (e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2); 2 mm or less (e.g., 1.95, 1.85, 1.75, 1.65, 1.55, 1.45, 1.35, 1.25, 1.15, 1.05, 0.95, 0.85, 0.75, 0.65, 0.55, 0.45, 0.35, 0.25, 0.15); or (e.g., 0.2-1.92; 0.3-1.82; 0.4-1.72; 0.5-1.62; 0.6-1.52; 0.7-1.42; 0.8-1.32; 0.9-1.22; 1-1.12; 1 mm).

Embodiment 16. The medical blade of any one of the preceding embodiments, wherein the symmetrical arcuate portion comprises a thickness between the concave side and the convex side, wherein the thickness is 0.01 mm or greater (e.g., 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71. 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2); 2 mm or less (e.g., 2, 1.9, 1.8, 1.7, 1.6, 1.5, 1.4, 1.3, 1.2, 1.1, 1, 0.99, 0.98, 0.97, 0.96, 0.95, 0.94, 0.93, 0.92, 0.91, 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.32, 0.31, 0.3, 0.29, 0.28, 0.27, 0.26, 0.25, 0.24, 0.23, 0.22, 0.21, 0.2, 0.19, 0.18, 0.02, 0.03, 0.04, 0.05, 0.06, 0.07, 0.08, 0.09, 0.1, 0.11, 0.12, 0.13, 0.14, 0.15, 0.16, 0.17, 0.16, 0.02, 0.03, 0.04, 0.05, 0.06, 0.07, 0.08, 0.09, 0.1, 0.11, 0.12, 0.13, 0.14, 0.15, 0.15, 0.14, 0.13, 0.12, 0.11, 0.1, 0.09, 0.08, 0.07, 0.06, 0.05, 0.04, 0.03, 0.02, 0.01); or 0.01 mm-2 mm (e.g., 0.02-1.9, 0.03-1.8, 0.04-1.7, 0.05-1.6, 0.06-1.5, 0.07-1.4, 0.08-1.3, 0.09-1.2, 0.1-1.1, 0.11-1, 0.12-0.99, 0.13-0.98, 0.14-0.97, 0.15-0.96, 0.16-0.95, 0.17-0.94, 0.18-0.93, 0.19-0.92, 0.2-0.91, 0.21-0.9, 0.22-0.89, 0.23-0.88, 0.24-0.87, 0.25-0.86, 0.26-0.85, 0.27-0.84, 0.28-0.83, 0.29-0.82, 0.3-0.81, 0.31-0.8, 0.32-0.79, 0.33-0.78, 0.34-0.77, 0.35-0.76, 0.36-0.75, 0.37-0.74, 0.38-0.73, 0.39-0.72, 0.4-0.71, 0.41-0.7, 0.42-0.69, 0.43-0.68, 0.44-0.67, 0.45-0.66, 0.46-0.65, 0.47-0.64, 0.48-0.63, 0.49-0.62, 0.5-0.61, 0.51-0.6, 0.52-0.59, 0.53-0.58, 0.54-0.57, 0.55-0.56).

Embodiment 17. The medical blade of any one of the preceding embodiments, wherein the width comprises 0.2 cm and the rise comprises 0.2 cm.

Embodiment 18. The medical blade of any one of the preceding embodiments, wherein the width comprises 0.6 cm and the rise comprises 0.6 cm.

Embodiment 19. The medical blade of any one of the preceding embodiments, wherein the width comprises 0.6 cm and the rise comprises 0.3 cm.

Embodiment 20. The medical blade of any one of the preceding embodiments, wherein the width comprises 1 cm and the rise comprises 0.2 cm.

Embodiment 21. The medical blade of any one of the preceding embodiments, wherein the width comprises 1 cm and the rise comprises 0.5 cm.

Embodiment 22. The medical blade of any one of the preceding embodiments, wherein the width comprises 1 cm and the rise comprises 1 cm.

Embodiment 23. The medical blade of any one of the preceding embodiments, wherein the width comprises 2 cm and the rise comprises 0.2 cm.

Embodiment 24. The medical blade of any one of the preceding embodiments, wherein the width comprises 3 cm and the rise comprises 0.2 cm.

Embodiment 25. The medical blade of any one of the preceding embodiments, wherein the length of the arc of the symmetrical arcuate portion comprises 1.4 mm or greater (e.g., 1.75, 2.25, 2.75, 3.25, 3.75, 4.25, 4.75, 5.25, 5.75, 6.25, 6.75, 7.25, 7.75, 8.25, 8.75, 9.25, 9.75, 10.25, 10.75, 15.5, 22, 27, 32, 37, 42, 47, 52, 57, 62, 67, 72, 77, 82, 87, 92, 97, 102, 107, 112, 117, 122, 127, 132, 137); 140 or less (e.g., 138, 134, 128, 124, 118, 114, 108, 104, 98, 94, 88, 84, 78, 74, 68, 64, 58, 54, 48, 44, 38, 34, 28, 24, 18, 14, 8, 4, 2.8, 2.4, 1.8, 1.4); or ranges from 1.4 mm to 139 mm (e.g., 1.75, 2.25, 2.75, 3.25, 3.75, 4.25, 4.75, 5.25, 5.75, 6.25, 6.75, 7.25, 7.75, 8.25, 8.75, 9.25, 9.75, 10.25, 10.75, 15.5, 22, 27, 32, 37, 42, 47, 52, 57, 62, 67, 72, 77, 82, 87, 92, 97, 102, 107, 112, 117, 122, 127, 132, 137).

Embodiment 26. The medical blade of any one of the preceding embodiments, wherein the area of the symmetrical arcuate portion comprises 0.26 mm² or greater (e.g., 0.36, 0.46, 0.56, 0.66, 0.76, 0.86, 0.96, 1.6, 2.6, 3.6, 4.6, 5.6, 6.6, 7.6, 8.6, 9.6, 10.6, 11.6, 12.6, 13.6, 14.6, 15.6, 26, 36, 46, 56, 66, 76, 86, 96, 106, 116, 126, 136, 146, 156, 166, 176, 186, 196, 206, 216, 226, 236, 246, 256); 263 mm² or less (e.g., 260, 250, 240, 230, 220, 210, 200, 190, 180, 170, 160, 150, 140, 130, 120, 110, 100, 90, 80, 70, 60, 50, 40, 30, 20, 10, 5, 4, 3, 2, 1, 0.5, 0.4, 0.3); or ranges from 0.26 mm²-263 mm² (e.g., 0.3-260, 0.5-255, 1-250, 1.5-245, 2-240, 2.5-235, 3-230, 3.5-225, 4-220, 2.5-215, 3-210, 3.5-200, 4-295, 4.5-290, 5-285, 5.5-280, 6-275, 6.5-270, 7-265, 7.5-260, 8-255, 8.5-250, 9-245, 9.5-240, 10-235, 15-230, 20-225, 25-220, 30-215, 35-210, 40-205, 45-200, 50-195, 55-190, 60-185, 65-180, 70-175, 75-170, 80-165, 85-160, 90-155, 95-150, 100-145, 105-140, 110-135, 115-130, 120-125).

Embodiment 27. The medical blade of any one of the preceding embodiments, wherein the medical blade is composed of a material selected from the group consisting of: metal (e.g., stainless steel, high carbon steel, tempered steel), ceramic (e.g., zirconium, zirconium dioxide), titanium, diamond, sapphire, obsidian, combinations of metals, and metal ceramic alloys (e.g., cermet).

Embodiment 28. The medical blade of any one of the preceding embodiments, wherein the medical blade is disposable (e.g., single use).

Embodiment 29. A scalpel, comprising: the medical blade of any one of embodiments 1-28; and a handle, wherein the handle is linear (e.g., elongated, essentially linear) along a longitudinal axis, wherein the handle comprises a first end (proximal) and a second end (distal), wherein the second end of the handle is fixedly attached (e.g., temporarily, permanently; non-folding, non-sliding) to the medical blade at the attachment region (wherein the second end of the handle is fixedly attached at the attachment region of the medical blade).

Embodiment 30. The scalpel of embodiment 29, wherein the handle is selected from the group consisting of: a flat handle, a cylindrical handle (e.g., resembling a pen), and an ergonomic handle.

Embodiment 31. The scalpel of any one of one of embodiments 29-30, wherein the second end of the handle comprises at least one projection (e.g., stud, screw, pin, elongated projection) that is configured to fasten in the at least one aperture of the medical blade.

Embodiment 32. The scalpel of embodiment 29, wherein the scalpel is disposable (i.e., one time use).

Embodiment 33. The scalpel of any one of embodiments 29-32, wherein the scalpel is for obtaining a skin sample from a subject.

Embodiment 34. Use of the medical blade of any one of embodiments 1-28 or the scalpel of any one of embodiments 29-33, for obtaining a skin sample.

Embodiment 35. Use of the medical blade of any one of embodiments 1-28 for obtaining a skin sample.

Embodiment 36. Use of the scalpel of any one of embodiments 29-33 for obtaining a skin sample.

Embodiment 37. A method of obtaining a skin sample from a subject, comprising: moving the cutting portion of any one of embodiments 1-36 through the surface of skin of a subject to obtain a skin sample.

Embodiment 38. The method of embodiment 37, wherein the moving occurs around the longitudinal axis.

Embodiment 39. The method of embodiment 37, wherein the moving occurs around an axis perpendicular to the longitudinal axis.

Embodiment 40. The method of any one of embodiments 37-39, wherein the moving comprises a downward motion.

Embodiment 41. The method of any one of embodiments 37-39, wherein the moving comprises a downward motion and an upward motion.

Embodiment 42. The method of any one of embodiments 37-39, wherein the moving comprises moving the cutting portion of the scalpel of any one of embodiments 29-33 in a downward and an upward motion to remove a skin sample from the subject.

Embodiment 43. The method of any one of embodiments 37-39, wherein the moving comprises scraping the cutting portion across and through a portion of the skin surface.

Embodiment 44. The method of any one of embodiments 37-41, wherein the symmetrical arcuate portion comprising a width of 1 cm and a rise of 0.1 cm, wherein the skin sample comprises a 0.6 cm diameter.

Embodiment 45. The method of any one of embodiments 37-41, wherein the symmetrical arcuate portion comprising a width of 1 cm and a rise of 0.2 cm, wherein the skin sample comprises a 0.8 cm diameter.

Embodiment 46. The method of any one of embodiments 37-41, wherein the symmetrical arcuate portion comprising a width of 0.6 cm and a rise of 0.1 cm, wherein the skin sample comprises a 0.45 cm diameter.

Embodiment 47. The method of any one of embodiments 37-41, wherein the symmetrical arcuate portion comprising a width of 0.6 cm and a rise of 0.2 cm, wherein the skin sample comprises a 0.57 cm diameter.

Embodiment 48. The method of any one of embodiments 37-41, wherein the symmetrical arcuate portion comprising a width of 0.6 cm and a rise of 0.3 cm, wherein the skin sample comprises a 0.6 cm diameter.

Embodiment 49. The method of any one of embodiments of 37-41, wherein the symmetrical arcuate portion comprising a width of 0.5 cm and a rise of 0.1 cm, wherein the skin sample comprises a 0.4 cm diameter.

Embodiment 50. The method of any one of embodiments of 37-41, wherein the symmetrical arcuate portion comprising a width of 0.5 cm and a rise of 0.2 cm, wherein the skin sample comprises a 0.49 cm diameter.

Embodiment 51. A scalpel, comprising:
(a) a medical blade comprising: a cutting portion, wherein the cutting portion comprises an asymmetrical arcuate portion, wherein the asymmetrical arcuate portion comprises a first end of the asymmetrical arcuate portion, a second end of the asymmetrical arcuate portion, and an arc therebetween, wherein the first end of the asymmetrical arcuate portion and the second end of the asymmetrical arcuate portion are on an axis (w-w axis), wherein the axis is the same axis, wherein the second end of the asymmetrical arcuate portion is distal to the first end of the asymmetrical arcuate portion, wherein the asymmetrical arcuate portion comprises a width extending on the axis between the first end of the asymmetrical arcuate portion and the second end of the asymmetrical arcuate portion, wherein the arc comprises a rise extending from a point along the axis between the first end of the asymmetrical arcuate portion and the second end of the asymmetrical arcuate portion to the furthest point of the arc (where the location of the point of the arc is configured to provide the greatest or longest length of the rise), wherein the symmetrical arcuate portion comprises: (i) a convex side; (ii) a concave side; (iii) atop edge; and (iv) a bottom edge, wherein the bottom edge comprises a cutting edge, wherein the asymmetrical arcuate portion comprises a height extending between the top edge and the bottom edge, wherein the cutting portion is configured to be fixed (or fixedly configured, operatively connectable) at the first end of the asymmetrical arcuate portion; and
(b) a handle,
   wherein the handle is linear (e.g., elongated, essentially linear) along a longitudinal axis, wherein the handle comprises a first end (proximal) and a second end (distal), wherein the second end of the handle is fixedly attached (e.g., temporarily, permanently; non-folding, non-sliding) to the medical blade at the attachment region (wherein the second end of the handle is fixedly attached at the attachment region of the medical blade).

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

1. A medical blade, comprising:
a cutting portion,
wherein the cutting portion comprises a symmetrical arcuate portion,
wherein the symmetrical arcuate portion comprises a first end of the symmetrical arcuate portion, a second end of the symmetrical arcuate portion, and an arc therebetween,
wherein the first end of the symmetrical arcuate portion and the second end of the symmetrical arcuate portion are on an axis,
wherein the axis is the same axis,
wherein the second end of the symmetrical arcuate portion is distal to the first end of the symmetrical arcuate portion,
wherein the symmetrical arcuate portion comprises a width extending on the axis between the first end of the symmetrical arcuate portion and the second end of the symmetrical arcuate portion,
wherein the arc comprises a rise extending from the center of the width to the center of the arc,
wherein the symmetrical arcuate portion comprises:
(i) a convex side;
(ii) a concave side;
(iii) a top edge; and
(iv) a bottom edge,
wherein the bottom edge comprises a cutting edge,
wherein the symmetrical arcuate portion comprises a height extending between the top edge and the bottom edge,
wherein the cutting portion is configured to be fixed at the first end of the symmetrical arcuate portion.

2. The medical blade of claim 1, further comprising:
a body portion,
wherein the body portion comprises a first end and a second end,
wherein the body portion is linear, extending along a longitudinal axis between the first end of the body portion and the second end of the body portion,
wherein the second end of the body portion is distal to the first end of the body portion,
wherein the cutting portion is configured to be fixed at the second end of the body portion,
wherein the body portion is configured to be fixed at the first end of the body portion.

3. The medical blade of claim 1, wherein the medical blade is a surgical blade.

4. The medical blade of claim 1, wherein the medical blade comprises a shape is selected from the group consisting of: a parabolic shape, a semi-elliptical shape, a semi-circular shape, a semi-ovoid shape, a C-shape, a J-shape, and a question mark shape.

5. The medical blade of claim 1, wherein the axis of the width extending between the first end of the symmetrical arcuate portion and the second end of the symmetrical arcuate portion is a longitudinal axis.

6. The medical blade of claim 1, wherein the axis of the width extending between the first end of the symmetrical arcuate portion and the second end of the symmetrical arcuate portion is a perpendicular to the longitudinal axis.

7. The medical blade of claim 1, wherein the first end of the symmetrical arcuate portion comprises an attachment region.

8. The medical blade of claim 2, wherein the first end of the body portion comprises an attachment region.

9. The medical blade of claim 7, wherein the attachment region comprises at least one aperture.

10. The medical blade of claim 9, wherein the attachment region is configured for attachment to a handle.

11. The medical blade of claim 1, wherein the width comprises 0.5 mm or greater.

12. The medical blade of claim 1, wherein the rise comprises 0.5 mm or greater.

13. The medical blade of claim 1, wherein the height comprises 0.1 mm or greater.

14. The medical blade of claim 1, wherein the symmetrical arcuate portion comprises a thickness between the concave side and the convex side, wherein the thickness is 0.01 mm or greater.

15. The medical blade of claim 1, wherein the medical blade is composed of a material selected from the group consisting of: metal, ceramic, titanium, diamond, sapphire, obsidian, combinations of metals, and metal ceramic alloys.

16. A scalpel, comprising:
the medical blade of claim 1; and
a handle,
wherein the handle is linear along a longitudinal axis,
wherein the handle comprises a first end and a second end,
wherein the second end of the handle is fixedly attached to the medical blade at the attachment region.

17. The scalpel of claim 16, wherein the second end of the handle comprises at least one projection that is configured to fasten in the at least one aperture of the medical blade.

18. A method of obtaining a skin sample from a subject, comprising:
moving the cutting portion of claim 16 through the surface of skin of a subject to obtain a skin sample.

19. The method of claim 18, wherein the moving occurs around the longitudinal axis or around an axis perpendicular to the longitudinal axis.

20. The method of claim 18, wherein the moving comprises moving the cutting portion in a downward and an upward motion to remove a skin sample from the subject.
